(19) United States
(12) Patent Application Publication
(10) Pub. No.: US 2023/0190629 A1
(43) Pub. Date: Jun. 22, 2023

(54) LONG-WEAR COMPOSITIONS CONTAINING ACID-FUNCTIONAL SILICONE ELASTOMER AND BLOCK COPOLYMER

(71) Applicant: L'OREAL, Paris (FR)

(72) Inventors: Mariko HASEBE, West New York, NJ (US); Hy Si BUI, Piscataway, NJ (US)

(73) Assignee: L'OREAL, Paris (FR)

(21) Appl. No.: 17/557,638

(22) Filed: Dec. 21, 2021

(51) Int. Cl. A61K 8/893; A61Q 1/00; A61K 8/06; A61K 8/90

(52) U.S. Cl. CPC A61K 8/893; A61Q 1/00; A61K 8/06; A61K 8/90; A61K 2800/31

(57) ABSTRACT

Compositions including at least one acid-functional silicone elastomer and at least one block copolymer, as well as methods of making such compositions and methods of applying such compositions to keratinous material are provided.

### FIELD OF THE INVENTION

The present invention relates to compositions comprising at least one acid-functional silicone elastomer and at least one block copolymer. Among other improved or beneficial properties, these compositions have surprisingly good stability, long-wear, adhesion, and/or transfer-resistance properties.

### DISCUSSION OF THE BACKGROUND

Many cosmetic compositions, including pigmented cosmetics such as foundations, mascaras and lipsticks, have been formulated in an attempt to possess long wearing properties upon application. Unfortunately, many of these compositions do not generally possess both good long-wear/transfer-resistance properties as well as good application properties, good comfort properties and/or good appearance properties (for example, matte or shine properties).

For example, with respect to lip compositions, commercial products containing silicon resins such as MQ resins are known. Such products are known to provide good long wear properties and/or transfer-resistance. However, such products possess poor application properties and/or poor feel upon application (for example, feels rough). Therefore, a second composition (topcoat) is separately applied to such products to improve poor properties of the compositions to make the products acceptable to consumers. Furthermore, the topcoat composition must be reapplied continually so that the product remains acceptable to consumers, meaning that the products are effectively not “long-wearing” as they require constant maintenance and reapplication.

Also, with respect to foundations, such products can provide good long wear properties and/or transfer-resistance. However, such long-wearing/transfer-resistant products can possess poor application and/or feel upon properties application, as well as poor appearance properties.

Compositions containing block copolymer are known. For example, U.S. Pat. No. 8,778,323, US 2007/0258923, US 2014/0105838, EP 2,707,404, WO 2012/156630, US 2014/0105838, WO 2013/190708, CN 104619307, EP 2,863,871, US 2015/0182440, and WO 2013/191303 may be relevant.

Further, compositions containing silicone elastomers are known. For example, U.S. Pat. Nos. 8,987,373, 9,150,703, 9,150,726, 9,175,139, 9,181,433, 9,200,146, US 2015/0073059, WO 2012/098116, US 2007/0053859, U.S. Pat. Nos. 7,790,148, 8,697,039, US 2010/0297050, U.S. Pat. No. 7,378,103, CN1781476, EP 1,579,845, US 2005/0220728 and EP 2,665,522 may be relevant.

However, there remains a need for improved compositions having improved cosmetic properties, particularly good stability, wear, transfer-resistance, adhesion, feel and/or appearance characteristics upon application.

Accordingly, one aspect of the present invention is a care and/or makeup and/or treatment composition for keratinous materials which has good cosmetic properties such as, for example, good stability, wear, transfer-resistance, adhesion, feel and/or appearance properties upon application.

### SUMMARY OF THE INVENTION

The present invention relates to compositions comprising at least one acid-functional silicone elastomer and at least one block copolymer.

The present invention also relates to emulsion compositions comprising water, at least one acid-functional silicone elastomer and at least one block copolymer.

The present invention also relates to anhydrous compositions comprising at least one acid-functional silicone elastomer and at least one block copolymer.

The present invention also relates to colored compositions comprising at least one coloring agent, at least one acid-functional silicone elastomer and at least one block copolymer.

The present invention also relates to methods of treating, caring for and/or making up keratinous materials such as, for example, lips, skin or eyelashes, by applying compositions of the present invention to a keratinous material in an amount sufficient to treat, care for and/or make up the keratinous material.

The present invention also relates to methods of enhancing the appearance of keratinous materials such as, for example, lips, skin or eyelashes, by applying compositions of the present invention to a keratinous material in an amount sufficient to enhance the appearance of the keratinous material.

It is to be understood that both the foregoing general description and the following detailed description are exemplary and explanatory only, and are not restrictive of the invention.

### DETAILED DESCRIPTION OF THE INVENTION

As used herein, the expression “at least one” means one or more and thus includes individual components as well as mixtures/combinations.

Other than in the operating examples, or where otherwise indicated, all numbers expressing quantities of ingredients and/or reaction conditions are to be understood as being modified in all instances by the term “about,” meaning within 10% of the indicated number.

“Film former” or “film forming agent” as used herein means any material such as, for example, a polymer or a resin that leaves a film on the substrate to which it is applied.

“Polymer” as used herein means a compound which is made up of at least two monomers.

“Keratinous materials” includes materials containing keratin such as hair, skin, eyebrows, lips and nails.

“Substituted” as used herein (unless otherwise indicated), means comprising at least one substituent. Non-limiting examples of substituents include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, hydroxyalkyl groups, and polysiloxane groups. The substituent(s) may be further substituted.

“Volatile”, as used herein, means having a flash point of less than about 100° C.

“Non-volatile”, as used herein, means having a flash point of greater than about 100° C.

“Anhydrous” means the compositions contain less than 1% water. Preferably, the compositions of the present invention contain less than 0.5% water, and preferably no water.

“Transfer” as used herein refers to the displacement of a fraction of a composition which has been applied to a keratinous material by contact with another substrate, whether of the same nature or of a different nature. For example, when a composition such as an eyeshadow, eyeliner or mascara has been applied, the composition can be transferred onto hands by rubbing or by contact of the hands with the eyes. By way of further example, when a composition such as a lipstick has been applied, the composition can be transferred onto teeth or hands, or onto the cheek of another person. Irrespective of composition type, the composition can also transfer from the keratinous material to which it has been applied to another substrate such as napkins, collars, glasses, cups or other containers.

“Transfer-resistance” as used herein refers to the quality exhibited by a composition in resisting transfer. To determine transfer-resistance, the amount of composition transferred from a keratinous material to a substrate may be evaluated and compared. For example, a composition may be transfer-resistant if, after application to a keratinous material such as lips, skin or eyelashes and contact with a substrate, a majority of the composition is left on the wearer. Further, the amount transferred may be compared with that transferred by other compositions, such as commercially-available compositions. In a preferred embodiment of the present invention, little or no composition is transferred to the substrate from the keratinous material.

“Long wear” compositions as used herein refers to compositions where the compositions, after application to a keratinous material, do not transfer or smudge after contact with another substrate and retain a consistent appearance on the keratinous material for an extended period of time. “Long wear” compositions, as used herein can also refer to compositions where color remains the same or substantially the same as at the time of application, as viewed by the naked eye, after an extended period of time. Long wear properties may be evaluated by any method known in the art for evaluating such properties. For example, long wear may be evaluated by a test involving the application of a composition to keratinous materials such as skin, eyelashes or lips and evaluating the color of the composition after an extended period of time. For example, the color of a composition may be evaluated immediately following application to the keratinous material and these characteristics may then be re-evaluated and compared after a certain amount of time. Further, these characteristics may be evaluated with respect to other compositions, such as commercially available compositions. Additionally, long wear properties may be evaluated by applying a sample, allowing it to dry, and then abrading the sample to determine removal/loss of sample.

The composition of the present invention may be in any form, either liquid or non-liquid (semi-solid, soft solid, solid, etc.). For example, it may be a paste, a solid, a gel, or a cream. It may be an emulsion, such as an oil-in-water or water-in-oil emulsion, a multiple emulsion, such as an oil-in-water-in-oil emulsion or a water-in-oil-in-water emulsion, or a solid, rigid or supple gel. The composition of the invention may, for example, comprise an external or continuous fatty phase. The composition can also be a molded composition or cast as a stick or a dish.

The compositions and methods of the present invention can comprise, consist of, or consist essentially of the essential elements and limitations of the invention described herein, as well as any additional or optional ingredients, components, or limitations described herein or otherwise useful in personal care.

In accordance with the present invention, compositions comprising at least one acid-functional silicone elastomer and at least one block copolymer are provided.

When the compositions are foundations, the compositions preferably have improved or beneficial non-transfer properties against water, sweat, sebum, saliva and/or oil, matity properties, and/or comfort upon application.

When the compositions are mascaras, the compositions preferably have improved or beneficial non-transfer properties against water, sweat, sebum, saliva and/or oil, shine properties, and/or glide upon application.

When the compositions are lip compositions, the compositions preferably have improved or beneficial non-transfer properties against water, sweat, sebum, saliva and/or oil, and/or shine properties.

Acid-Functional Silicone Elastomer

In accordance with the present invention, compositions comprising at least one acid-functional silicone elastomer are provided. An “acid-functional silicone elastomer” is a compound which comprises a siloxy (Si—O) group bonded to another moiety having acid functional group(s), preferably carboxy functional group(s). Acid-functional silicone elastomers are described or referenced in U.S. patent application 2016/0199286, the entire contents of which is hereby incorporated by reference.

Preferably, the acid-functional elastomer has a general formula: A-B-A or B-A-B, wherein each A independently comprises a polysiloxane moiety having at least two siloxy (Si—O) groups and wherein each B independently comprises a moiety, or a precursor thereof, having at least two carboxyl groups and wherein B is bonded to a silicon atom in A.

According to preferred embodiments, the acid-functional elastomer has at least two siloxy (Si—O) groups and at least two carboxyl groups and is the reaction product of a reaction of a first component having at least one reactive group; a second component having at least one reactive group; and a third component having at least two reactive groups reactive with the reactive groups of the first and second component for linking the first component to the second component.

According to preferred embodiments, the at least one reactive component of the first component and the second component are independently selected from a hydroxyl group or an amine group and wherein the at least two reactive groups of the third component are anhydride groups. In more specific related embodiments, the first and second component are selected from siloxanes having at least one hydroxyl group or organic alcohols having at least one hydroxyl group and the third component a siloxane having at least two terminal anhydride groups. In other more specific related embodiments, the first and second component are selected from siloxanes having at least one amine group or organic amines having at least one amine group and the third component a siloxane having at least two pendant anhydride groups.

According to preferred embodiments, the at least one reactive component of the first component and the second component are anhydride groups and wherein the at least two reactive groups of the third component are hydroxyl groups or amine groups. In more specific related embodiments, the first and second components are siloxanes and the anhydride groups of these first and second components are pendant. In certain of these embodiments, the third component is selected from i) an organic polyol having at least two hydroxyl groups; ii) a third siloxane having at least two hydroxyl groups; iii) an organic polyamine having at least two amine groups; or iv) a third siloxane having at least two amine groups.

According to preferred embodiments, each of the first and second reactive components can be chemically (or physically) the same, such as two separate molecules of the same reactive component (or type). For example, the first and second reactive components can be provided together, such as in an “A-part” (or A-side) of a system for forming the elastomer of the present invention. Alternatively, the first and second reactive components can be provided separately, especially when they are different from each other. This may be useful for formulation purposes. However, separation is not required, as the first and second reactive components are typically inert with respect to each other.

The third reactive component can be provided separate from the first and second reactive component, such as in a “B-part” (or B-side) of a system for forming the elastomer of the present invention. If the elastomer, or cosmetic composition, includes one or more optional additives, the additive(s) can be included with either of, each of, or a combination of, the system parts. The system may include more than two parts. Optionally, various types of conventional additives can be utilized depending, for example, on the end use of cosmetic composition. The present invention is not limited to any particular arrangement of the system, or to any particular additive or additives.

According to preferred embodiments, the siloxanes used to form the elastomers of the present invention consist of siloxane bonds (Si—O—Si) within each of their backbones. Alternatively, each of the siloxanes may include siloxane bonds separated by one or more bivalent groups, e.g. a —CH₂— linking group. Further examples of suitable bivalent groups include polyether groups, e.g. a —CH₂CH₂O— linking group (i.e., an EO group), a —CH(CH₃)CH₂O— linking group (i.e., a PO group), etc. Combinations of different bivalent groups may be present within each of their backbones. Each of the bivalent groups may be singular or repeated, e.g. 2 times, 5 times, 10 times, >10 times, etc. In certain embodiments, the first and second siloxanes are free of polyether groups.

According to preferred embodiments, each of the siloxanes comprise at least one [SiR₂—O-] unit (D or R₂SiO_{2/2} units). Typically, each of the siloxanes has repeating D units, which generally constitute linear portions of the siloxanes. The siloxanes also typically have terminal R₃SiO_{1/2} units (M units). In these embodiments, R is an independently selected substituted or unsubstituted hydrocarbyl group.

According to preferred embodiments, each of the siloxanes described herein may optionally be branched, partially branched, and/or may include a resinous portion having a three-dimensional networked structure. In such embodiments, the respective siloxane may further comprise RsiO_{3/2} units (T units) and/or SiO_{4/2} units (Q units). Branching of the siloxane itself, or the resinous portion of the siloxane, if present, can be attributable to the presence of T and/or Q units. Branching may also be attributable to side groups of one or more D units. In various embodiments, the siloxanes are free of T units, Q units, or both T and Q units. Where more than one siloxane is described, the siloxanes can be the same or different, e.g. one is linear and one is branched, both are branched, both are linear, etc.

According to preferred embodiments, the acid-functional elastomer has the general formula:

R⁵₃Si—O—[SiR¹R⁴—O—]_{w}[SiR¹R²—O—]ₓ[SiR¹R⁴—O—]_{y}SiR⁵₃

or

(CH₃)₃Si—O—[Si(CH₃)₂O—]_{w}[Si(CH₃)R²—O—]ₓ[Si(CH₃)R⁴—O—]_{y}Si(CH₃)₃

wherein R¹ is an independently selected substituted or unsubstituted hydrocarbyl group. By “substituted” in this context, it is meant that one or more hydrogen atoms of the hydrocarbon may be replaced with atoms other than hydrogen (e.g. a halogen atom), or a carbon atom within the chain of R¹ may be replaced with an atom other than carbon, i.e., R¹ may include one or more heteroatoms within the chain, such as oxygen, sulfur, nitrogen, etc. Examples of suitable hydrocarbyl groups represented by R¹ include alkyl, aryl, alkenyl, alkaryl, and aralkyl, groups. Preferably, R¹ is an independently selected alkyl group having from 1 to 20, 1 to 15, 1 to 10, 1 to 6, 1 to 4, or 1 to 2, carbon atoms, or any number of carbon atoms in between. Specific examples of suitable alkyl groups as R¹ include methyl groups, ethyl groups, propyl groups, butyl groups, pentyl groups, etc. Typically, R¹ is a methyl group (i.e., —CH₃).

R² is preferably a pendant anhydride group of the following general formula (A):

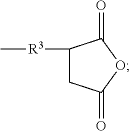

where R³ is a divalent group. Preferably, R³ is a hydrocarbylene, heterohydrocarbylene, or organoheterylene group, with R³ preferably being (CH₂)ₙ where n is an integer selected from 1 to 30, 1 to 25, 1 to 20, 1 to 15, 1 to 10, 1 to 5, or 1 to 3, or any number in between. Most preferably, n is 3.

Each of R⁴ and R⁵ can be an independently selected substituted or unsubstituted hydrocarbyl group. Examples of suitable groups for each of R⁴ and R⁵ are as described for R¹. In certain embodiments, each R⁴ is an independently selected alkyl group, aryl group, or (R⁶O)ₘ group. If R⁴ is a (R⁶O)ₘ group, R⁶ is typically an alkyl group or aryl group and m is an integer selected from 1 to 50, 1 to 25, 1 to 10, 1 to 5, or 1, or any number in between. The (R⁶O)ₘ group may also be referred to as a polyether group. In certain embodiments, R⁴ is an independently selected alkyl group having from 2 to 20, 2 to 15, 2 to 10, 2 to 5, or 2, carbon atoms, or any number of carbon atoms in between. In some embodiments, R⁴ may be silicone side chain of the siloxane. The groups represented by subscripts w, x, and y, may be present in any order within the respective siloxane, including a different order than that which is represented above. Moreover, these groups may be present in randomized or block form. Preferably, R⁴ is either an alkyl group or a polyether group.

Preferably, w is an integer selected from zero (0) to 1,000, 0 to 950, 0 to 750, 0 to 500, 0 to 400, 1 to 350, 1 to 300, 25 to 250, 50 to 200, 50 to 150, 75 to 125, 90 to 110, 90 to 100, or 90 to 95, or any number in between. In a specific embodiment, w is 93. Typically, x is an integer selected from 1 to 100, 1 to 75, 1 to 50, 1 to 25, 1 to 20, 1 to 10, or 1 to 5, or any number in between. In a specific embodiment, x is 3. Typically, y is an integer selected from 0 to 1,000, 0 to 950, 0 to 750, 0 to 500, 0 to 400, 1 to 350, 1 to 300, 1 to 250, 1 to 200, 1 to 150, 1 to 100, 1 to 75, 1 to 50, 1 to 25, 1 to 20, 1 to 15, 1 to 10, or 1 to 5, or any number in between.

According to preferred embodiments, w and y are not simultaneously 0. Said another way, in these embodiments, each of the first and second siloxanes include at least one D unit associated with each of the x units and at least one of the w and y units. In certain embodiments, the sum of w+x+y is from 25 to 1,500, 25 to 1,000, 25 to 900, 25 to 800, 25 to 700, 25 to 600, 25 to 500, 25 to 400, 25 to 300, 50 to 200, 75 to 150, 85 to 125, or 90 to 110, or any number in between. In these embodiments, x is at least 1, at least 10, at least 25, at least 50, at least 75, or at least 85. In this way, each of the first and second siloxanes has at least one of the pendant anhydride groups, and can have other side groups based on the presence of one or more D units associated with w and y.

The acid-functional silicone elastomer preferably is present in the compositions of the present invention in an active solid content amount ranging from about 0.5% to about 15%, preferably from about 0.75% to about 10%, and preferably from about 1% to about 6%, by weight with respect to the total weight of the composition, including all ranges and subranges therebetween. It is to be understood that acceptable ranges of acid-functional silicone elastomer present in invention compositions include 2%-15%, 2%-10%, 2%-6%, 2%-5%, 5%-15%, 5%-10%, and 5%-6% by weight based on the weight of the composition.

Block Copolymer

According to preferred embodiments of the present invention, compositions comprising at least one block copolymer are provided. The block copolymers of the present invention are characterized by the presence of at least one “hard” segment, and at least one “soft” segment. Aside from their compositional nature, the hard and soft segments of the block copolymers of the present invention are defined in terms of their respective glass transition temperatures, “T_{g}” More particularly, the hard segment has a T_{g} of about 50° C. or more, whereas the soft segment has a T_{g} of about 20° C. or less. The glass transition temperature T_{g} for the hard block can range from about 50° C. to about 150° C.; about 60° C. to about 125° C.; about 70° C. to about 120° C.; or about 80° C. to about 110° C. The glass transition temperature T_{g} for the soft segment of the block copolymer can range from about 20° C. to about −150° C.; about 0° C. to about −135° C.; about −10° C. to about −125° C.; and about −25° C. to about −100° C. A more in depth explanation can be found in U.S. Pat. Nos. 5,294,438 and 6,403,070, the entire contents of which are hereby incorporated by reference.

The block copolymer of the present invention is a thermoplastic elastomer. The hard segments of the thermoplastic elastomer typically comprise vinyl monomers in varying amounts. Examples of suitable vinyl monomers include, but are not limited to, styrene, methacrylate, acrylate, vinyl ester, vinyl ether, vinyl acetate, and the like.

The block copolymer of the present invention comprises at least one hard segment which is styrene.

The soft segments of the thermoplastic elastomer typically comprise olefin polymers and/or copolymers which may be saturated, unsaturated, or combinations thereof. Suitable olefin copolymers may include, but are not limited to, ethylene/propylene copolymers, ethylene/butylene copolymers, propylene/butylene copolymers, polybutylene, polyisoprene, polymers of hydrogenated butanes and isoprenes, and mixtures thereof.

Thermoplastic elastomers useful in the present invention include block copolymers e.g., di-block, tri-block, multi-block, radial and star block copolymers, and mixtures and blends thereof. A di-block thermoplastic elastomer is usually defined as an A-B type or a hard segment (A) followed by a soft segment (B) in sequence. A tri-block is usually defined as an A-B-A type copolymer or a ratio of one hard, one soft, and one hard segment. Multi-block or radial block or star block thermoplastic elastomers usually contain any combination of hard and soft segments, provided that the elastomers possess both hard and soft characteristics.

In preferred embodiments, the thermoplastic elastomer of the present invention may be chosen from the class of Kraton™ rubbers (Shell Chemical Company) or from similar thermoplastic elastomers. Kraton™ rubbers are thermoplastic elastomers in which the polymer chains comprise a di-block, tri-block, multi-block or radial or star block configuration or numerous mixtures thereof. The Kraton™ tri-block rubbers have polystyrene (hard) segments on each end of a rubber (soft) segment, while the Kraton™ di-block rubbers have a polystyrene (hard) segment attached to a rubber (soft) segment. The Kraton™ radial or star configuration may be a four-point or other multipoint star made of rubber with a polystyrene segment attached to each end of a rubber segment. The configuration of each of the Kraton™ rubbers forms separate polystyrene and rubber domains.

Each molecule of Kraton™ rubber is said to comprise block segments of styrene monomer units and rubber monomer and/or co-monomer units. The most common structure for the Kraton™ triblock copolymer is the linear A-B-A block type styrene-butadiene-styrene, styrene-isoprene-styrene, styrene-ethylenepropylene-styrene, or styrene-ethylenebutylene-styrene. The Kraton™ di-block is preferably the AB block type such as styrene-ethylenepropylene, styrene-ethylenebutylene, styrene-butadiene, or styrene-isoprene. The Kraton™ rubber configuration is well known in the art and any block copolymer elastomer with a similar configuration is within the practice of the invention. Other block copolymers are sold under the tradename Septon (which represent elastomers known as SEEPS, sold by Kurary, Co., Ltd) and those sold by Exxon Dow under the tradename Vector™.

Other thermoplastic elastomers useful in the present invention include those block copolymer elastomers comprising a styrene-butylene/ethylene-styrene copolymer (tri-block), an ethylene/propylene-styrene copolymer (radial or star block) or a mixture or blend of the two. (Some manufacturers refer to block copolymers as hydrogenated block copolymers, e.g. hydrogenated styrene-butylene/ethylene-styrene copolymer (tri-block)).

The amounts of the block (co)polymer or (co)polymers, as well as their structure (di-block, tri-block, etc.), affect the nature of the thermoplastic elastomer, including its gelled form, which may range from fragile to soft/flexible to firm. For instance, soft gels contain relatively high amounts of soft segments, and firm gels contain relatively high amounts of hard segments. The overall properties of the composition may also be affected by including more than one such block copolymer e.g., including a mixture of copolymers. For example, the presence of tri-block copolymers enhances the integrity of the film formed. The gel may also be transparent, translucent or opaque, depending upon the other cosmetically acceptable ingredients added, as described herein.

Although the block copolymers of the present invention comprise styrene, it is preferred that the styrene content of the block copolymer be less than 40% by weight, preferably less than 30% by weight, and more preferably less than 20% by weight, based on the weight of the block copolymer, and preferably greater than 5% by weight, preferably greater than 7% by weight, and preferably greater than 10% by weight, including all ranges and subranges therebetween such as, for example, 10%-30%, 6%-22%, 5%-40%, etc. This is because of the tendency of block copolymers having a styrene content of greater than 40% by weight to harden/gel in conventional carrier systems. However, in the event that a block copolymer having a styrene content of greater than 40% by weight is used, it may be necessary to also employ a co-solvent or functional ingredient capable of dissolving a styrene block in an amount effective to control the hardening/gelling of the styrene-containing compound in the cosmetic composition.

A particularly preferred block copolymer for use in the present invention is a combination of di-block and tri-block copolymers of styrene-ethylene/butylene-styrene (SEBS), commercially available from Shell Chemical Company under trade name Kraton G1657M. It should be noted, however, that any thermoplastic elastomer of the block copolymer type having at least one soft and at least one hard segment may be used without departing from the spirit of the invention.

The block copolymer will preferably have a solubility parameter, relative to the tackifier component, of δ±2, more preferably δ±1.7, more preferably δ±1.5, more preferably δ±1.3, more preferably δ±1.0, more preferably δ±0.7, more preferably δ±0.5, and more preferably δ±0.3.

The block copolymer preferably is present in the compositions of the present invention in an active solid content amount ranging from about 0.5% to about 30%, preferably from about 0.75% to about 20%, and preferably from about 1% to about 10%, by weight with respect to the total weight of the composition, including all ranges and subranges therebetween. According to preferred embodiments, compositions of the present invention contain at least about 2% by weight of the total weight of the composition, preferably at least about 5% by weight of the total weight of the composition, of the block copolymer. It is to be understood that acceptable ranges of block copolymer present in the invention compositions include 2%-30%, 2%-20%, 2%-10%, 2%-5%, 5%-30%, 5%-20%, and 5%-10% by weight based on the weight of the composition.

According to preferred embodiments, sufficient block copolymer is added to the composition of the present invention to increase strain sweep of the composition by an amount of at least 5 times as compared to the strain sweep of the same composition without the block copolymer (comparator), preferably at least 10 times the strain sweep of the comparator, preferably at least 20 times the strain sweep of the comparator, including ranges such as 5 to 50 times, 10 to 50 times, 20 to 50 times, 5 to 40 times, 10 to 40 times, 20 to 40 times, 5 to 33 times, 10 to 33 times, and 20 to 33 times, including all ranges and subranges therebetween, where strain sweep is determined per the methodology in example 3 or equivalent methodology known in the art.

While not wishing to be bound by any theory, it is believed that increasing strain sweep of the invention compositions by combining the block copolymer(s) and the acid-functional silicone elastomer(s) helps lead to increased adhesion to keratinous materials (for example, increased adhesion of a lip composition to lips or increased adhesion of a foundation to skin) and/or helps lead to increased phase stability of the composition (by providing homogenous dispersion of different phases of the composition (for example, oil and water phases of an emulsion) as determined visually and/or microscopically. In this regard, it is noted that for purposes of “consisting essentially of,” the basic and novel property is strain sweep.

According to preferred embodiments, the block copolymer and acid-functional silicone elastomer are present in the compositions of the present invention in an active solid content weight ratio of about 3:1 to about 1:1, preferably a weight ratio of about 2.5:1 to about 1:1, and preferably about 2:1 to about 1:1, including all ranges and subranges therebetween. Preferably, more block copolymer than acid-functional silicone elastomer is present in the compositions of the present invention.

According to preferred embodiments, the block copolymer and acid-functional silicone elastomer are present in the compositions of the present invention such that they are “miscible” with each other. In this context, “miscible” means that the block copolymer and silicone elastomer mix well. For example, in compositions containing pigments, “miscibility” can be determined visually: compositions in which pigments disperse such that streaks are not visually noticeable and/or compositions in which pigments cannot be visually detected on the composition's surface are compositions having “miscibility” of the block copolymer and acid-functional silicone elastomer. If streaks are present and pigments appear on the composition's surface, the block copolymer and the acid-functional silicone elastomer are not mixing well and are not “miscible.”

Tackifiers

According to preferred embodiments of the present invention, compositions of the present invention may optionally further comprise at least one tackifier. A substance is described as a tackifier if, by adding it to a block copolymer, the resulting composition has the properties of a pressure sensitive adhesive. In general, tackifiers can be divided into four different families in terms of their chemistry: hydrocarbon resins, terpenes, amorphous (i.e. non-crystalline) rosins, rosin esters and their derivatives, and pure monomer resins. These tackifiers are characterized by their compatibility with at least one segment of the block copolymer. By the term “compatible”, it is meant, for example, that when the block copolymer and tackifier are mixed, the combination of at least one segment of the block copolymer with the tackifier forms a polymer blend having a single glass transition temperature T_{g} which may be measured by DMA, DSC or neutron light scattering.

The compatibility of the block copolymer and the tackifier may also be defined in terms of solubility parameters. The solubility parameter δ according to the Hansen solubility space is defined in the article “Solubility Parameter Values” by Eric A. Grulke in the work “Polymer Handbook” 3^{rd} edition, Chapter VII, pages 519-559, the entire content of which is hereby incorporated by reference, by the relationship:

δ=(d_{D}²+d_{P}²+d_{H}²)^{1/2}, in which:
d_{D} characterizes the London dispersion forces resulting from the formation of dipoles induced during molecular impacts,
d_{P} characterizes the forces of Debye interactions between permanent dipoles,
d_{H} characterizes the forces of specific interactions (hydrogen bond, acid/base or donor/acceptor type and the like). The definition of the solvents in the three-dimensional solubility space according to Hansen is given in the article by C. M. Hansen: “The three-dimensional solubility parameters” J. Paint Technol., 39, 105(1967), the entire content of which is hereby incorporated by reference.

The at least one tackifier used in the present invention preferably has a solubility parameter corresponding to δ and the block copolymer preferably has at least one segment whose solubility parameter corresponds to δ±2, preferably δ±1.7, more preferably δ±1.5, more preferably δ±1.3, more preferably δ±1.0, more preferably δ±0.7, more preferably δ±0.5, and more preferably δ±0.3.

Examples of suitable tackifiers, include, but are not limited to, aliphatic hydrocarbon resins, aromatic modified aliphatic hydrocarbon resins, hydrogenated polycyclopentadiene resins, polycyclopentadiene resins, gum rosins, gum rosin esters, wood rosins, wood rosin esters, tall oil rosins, tall oil rosin esters, polyterpenes, aromatic modified polyterpenes, terpene phenolics, aromatic modified hydrogenated polycyclopentadiene resins, hydrogenated aliphatic resin, hydrogenated aliphatic aromatic resins, hydrogenated terpenes and modified terpenes, hydrogenated rosin acids, hydrogenated rosin esters, polyisoprene, partially or fully hydrogenated polyisoprene, polybutenediene, partially or fully hydrogenated polybutenediene, and the like. As is evidenced by some of the cited examples, the tackifier may be fully or partially hydrogenated. The tackifier may also be non-polar, where “non-polar” means that the tackifier is substantially free of monomers having polar groups. Preferably, polar groups are not present; however, if they are present, they are preferably present in an amount of up to about 5% by weight, preferably up to about 2% by weight, and more preferably up to about 0.5% by weight.

In preferred embodiments, the tackifier may have a softening point (Ring and Ball, as measured by ASTM E-28) of about 80° C. to about 150° C., preferably about 100° C. to about 130° C. In other preferred embodiments, the tackifier may be liquid and have an R and B softening point of between about −70° C. and about 70° C.

According to preferred embodiments, the tackifiers are hydrogenated hydrocarbon resins such as a hydrogenated styrene/methyl styrene/indene copolymer e.g., styrene/methyl styrene/indene copolymers which include R1090, R1100, R7100, S1100, and S5100, all which are commercially available from Eastman Chemical under the trade name Regalite®. In other embodiments, aliphatic or aromatic hydrocarbon-based tackifying resins, for instance the resins sold under the name “Piccotac” and “Hercotac” from Hercules or “Escorez” from Exxon, may also be used. It is also to be understood that mixtures of tackifiers may also be employed without departing from the spirit of the invention.

A particularly preferred tackifier for use in the present invention is a hydrogenated hydrocarbon resin such as, for example, a hydrogenated styrene/methyl styrene/indene copolymer, commercially available from Eastman under the tradename Regalite® R1100.

According to particularly preferred embodiments, the compositions of the present invention comprise an equivalent amount of the at least one block copolymer and the at least one tackifier, or more of the at least one block copolymer as compared to the at least one tackifier. For example, the at least one block copolymer and the at least one tackifier can be present in ratios of 1.25, 1.00, 0.75, 0.50, 0.33, 0.25, and 0.10, including all ranges and subranges therebetween, with ratios from 1.00 to 0.33 being particularly preferred.

If present in the compositions of the present invention, the tackifier(s) are preferably present in an amount of from about 0.1 to about 10 percent by weight, preferably from 1 to 10 percent by weight, preferably from 1 to 8 percent by weight and preferably from 1 to 5 percent by weight of the total weight of the composition, including all ranges and subranges therebetween

However, according to other preferred embodiments, the compositions of the present invention contain less than 0.1% tackifier(s), preferably less than 0.05% tackifier(s), and preferably no tackifier(s).

Coloring Agents

According to preferred embodiments of the present invention, compositions of the present invention may optionally further comprise at least one coloring agent. According to this embodiment, the coloring agent(s) is/are preferably chosen from pigments, dyes, nacreous pigments, pearling agents, and mixtures thereof.

Representative liposoluble dyes which may be used according to the present invention include Sudan Red, DC Red 17, DC Green 6, ß-carotene, soybean oil, Sudan Brown, DC Yellow 11, DC Violet 2, DC Orange 5, annatto, and quinoline yellow. The liposoluble dyes, when present, generally have a concentration ranging up to 40% by weight of the total weight of the composition, such as from 0.0001% to 30%, including all ranges and subranges therebetween.

The nacreous pigments which may be used according to the present invention may be chosen from colored nacreous pigments such as titanium mica with iron oxides, titanium mica with ferric blue or chromium oxide, titanium mica with an organic pigment chosen from those mentioned above, and nacreous pigments based on bismuth oxychloride. The nacreous pigments, if present, be present in the composition in a concentration ranging up to 50% by weight of the total weight of the composition, such as from 0.0001% to 40%, preferably from 0.001% to 30%, including all ranges and subranges therebetween.

The pigments, which may be used according to the present invention, may be chosen from white, colored, inorganic, organic, polymeric, and nonpolymeric pigments. Representative examples of mineral pigments include titanium dioxide, zirconium oxide, zinc oxide, cerium oxide, iron oxides, chromium oxides, manganese violet, ultramarine blue, chromium hydrate, and ferric blue. Representative examples of organic pigments include carbon black, pigments of D & C type, and lakes based on cochineal carmine, barium, strontium, calcium, and aluminum.

If present, the coloring agents may be present in the composition in a concentration ranging up to 50% by weight of the total weight of the composition, such as from 0.0001% to 40%, and further such as from 0.001% to 30%, including all ranges and subranges therebetween.

Oil Phase

According to preferred embodiments of the present invention, compositions further comprising at least one fatty substance are provided. Suitable fatty substances include oil(s) and/or wax(es). “Oil” means any non-aqueous medium which is liquid at ambient temperature (25° C.) and atmospheric pressure (760 mm Hg). A “wax” for the purposes of the present disclosure is a lipophilic fatty compound that is solid at ambient temperature (25° C.) and changes from the solid to the liquid state reversibly, having a melting temperature of more than 30° C. and, for example, more than 45° C., which can be as high as 150° C., a hardness of more than 0.5 MPa at ambient temperature, and an anisotropic crystalline organization in the solid state. By taking the wax to its melting temperature, it is possible to use wax(es) by themselves as carriers and/or it is possible to make wax(es) miscible with the oils to form a microscopically homogeneous mixture.

Suitable oils include volatile and/or non-volatile oils. Such oils can be any acceptable oil including but not limited to silicone oils and/or hydrocarbon oils.

According to certain embodiments, the compositions of the present invention preferably comprise one or more volatile silicone oils. Examples of such volatile silicone oils include linear or cyclic silicone oils having a viscosity at room temperature less than or equal to 6 cSt and having from 2 to 7 silicon atoms, these silicones being optionally substituted with alkyl or alkoxy groups of 1 to 10 carbon atoms. Specific oils that may be used in the invention include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils which may be used include KF 96A of 6 cSt viscosity, a commercial product from Shin Etsu having a flash point of 94° C. Preferably, the volatile silicone oils have a flash point of at least 40° C.

Non-limiting examples of volatile silicone oils are listed in Table 1 below.

TABLE 1

| | Flash Point | Viscosity |
|---|---|---|
| Compound | (° C.) | (cSt) |
| Octyltrimethicone | 93 | 1.2 |
| Hexyltrimethicone | 79 | 1.2 |
| Decamethylcyclopentasiloxane | 72 | 4.2 |
| (cyclopentasiloxane or D5) | | |
| Octamethylcyclotetrasiloxane | 55 | 2.5 |
| (cyclotetradimethylsiloxane or D4) | | |
| Dodecamethylcyclohexasiloxane (D6) | 93 | 7 |
| Decamethyltetrasiloxane(L4) | 63 | 1.7 |
| KF-96 A from Shin Etsu | 94 | 6 |
| PDMS (polydimethylsiloxane) DC 200 | 56 | 1.5 |
| (1.5 cSt) from Dow Corning | | |
| PDMS DC 200 (2 cSt) from Dow | 87 | 2 |
| Corning | | |

Further, a volatile linear silicone oil may be employed in the present invention. Suitable volatile linear silicone oils include those described in U.S. Pat. No. 6,338,839 and WO03/042221, the contents of which are incorporated herein by reference. In one embodiment the volatile linear silicone oil is decamethyltetrasiloxane. In another embodiment, the decamethyltetrasiloxane is further combined with another solvent that is more volatile than decamethyltetrasiloxane.

According to certain embodiments of the present invention, the composition of preferably comprises one or more non-silicone volatile oils and may be selected from volatile hydrocarbon oils, volatile esters and volatile ethers. Examples of such volatile non-silicone oils include, but are not limited to, volatile hydrocarbon oils having from 8 to 16 carbon atoms and their mixtures and in particular branched C₈ to C₁₆ alkanes such as C₈ to C₁₆ isoalkanes (also known as isoparaffins), isohexacecane, isododecane, isodecane, and for example, the oils sold under the trade names of Isopar or Permethyl. Preferably, the volatile non-silicone oils have a flash point of at least 40° C.

Non-limiting examples of volatile non-silicone volatile oils are given in Table 2 below.

TABLE 2

| Compound | Flash Point (° C.) |
|---|---|
| Isododecane | 43 |
| Propylene glycol n-butyl ether | 60 |
| Ethyl 3-ethoxypropionate | 58 |
| Propylene glycol methylether acetate | 46 |
| Isopar L (isoparaffin C₁₁-C₁₃) | 62 |
| Isopar H (isoparaffin C₁₁-C₁₂) | 56 |

The volatility of the solvents/oils can be determined using the evaporation speed as set forth in U.S. Pat. No. 6,338,839, the contents of which are incorporated by reference herein.

According to certain embodiments of the present invention, the composition comprises at least one non-volatile oil. Examples of non-volatile oils that may be used in the present invention include, but are not limited to, polar oils such as:
hydrocarbon-based plant oils with a high triglyceride content consisting of fatty acid esters of glycerol, the fatty acids of which may have varied chain lengths, these chains possibly being linear or branched, and saturated or unsaturated; these oils are especially wheat germ oil, corn oil, sunflower oil, karite butter, castor oil, sweet almond oil, macadamia oil, apricot oil, soybean oil, rapeseed oil, cottonseed oil, alfalfa oil, poppy oil, pumpkin oil, sesame seed oil, marrow oil, avocado oil, hazelnut oil, grape seed oil, blackcurrant seed oil, evening primrose oil, millet oil, barley oil, quinoa oil, olive oil, rye oil, safflower oil, candlenut oil, passion flower oil or musk rose oil; or caprylic/capric acid triglycerides, for instance those sold by the company Stearineries Dubois or those sold under the names Miglyol 810, 812 and 818 by the company Dynamit Nobel;
synthetic oils or esters of formula R₅COOR₆ in which R₅ represents a linear or branched higher fatty acid residue containing from 1 to 40 carbon atoms, including from 7 to 19 carbon atoms, and R₆ represents a branched hydrocarbon-based chain containing from 1 to 40 carbon atoms, including from 3 to 20 carbon atoms, with R₆+R₇≥10, such as, for example, Purcellin oil (cetostearyl octanoate), isononyl isononanoate, octyldodecyl neopentanoate, C₁₂ to C₁₅ alkyl benzoate, isopropyl myristate, 2-ethylhexyl palm itate, and octanoates, decanoates or ricinoleates of alcohols or of polyalcohols; hydroxylated esters, for instance isostearyl lactate or diisostearyl malate; and pentaerythritol esters;
synthetic ethers containing from 10 to 40 carbon atoms;
C₈ to C₂₆ fatty alcohols, for instance oleyl alcohol, cetyl alcohol, stearyl alcohol, and cetearyl alcohol; and
mixtures thereof.

Further, examples of non-volatile oils that may be used in the present invention include, but are not limited to, non-polar oils such as branched and unbranched hydrocarbons and hydrocarbon waxes including polyolefins, in particular Vaseline (petrolatum), paraffin oil, squalane, squalene, hydrogenated polyisobutene, hydrogenated polydecene, polybutene, mineral oil, pentahydrosqualene, and mixtures thereof.

According to preferred embodiments, if present, the at least one oil is present in the compositions of the present invention in an amount ranging from about 5 to about 80% by weight, more preferably from about 10 to about 70% by weight, and most preferably from about 15 to about 60% by weight, based on the total weight of the composition, including all ranges and subranges within these ranges.

According to preferred embodiments of the present invention, the compositions of the present invention further comprise at least one wax. Suitable examples of waxes that can be used in accordance with the present disclosure include those generally used in the cosmetics field: they include those of natural origin, such as beeswax, carnauba wax, candelilla wax, ouricoury wax, Japan wax, cork fibre wax or sugar cane wax, rice bran wax, rice wax, montan wax, paraffin wax, lignite wax or microcrystalline wax, ceresin or ozokerite, and hydrogenated oils such as hydrogenated castor oil or jojoba oil; synthetic waxes such as the polyethylene waxes obtained from the polymerization or copolymerization of ethylene, and Fischer-Tropsch waxes, or else esters of fatty acids, such as octacosanyl stearate, glycerides which are concrete at 30° C., for example at 45° C.

According to particularly preferred embodiments of the present invention, the compositions of the present invention further include at least one silicone wax. Examples of suitable silicone waxes include, but are not limited to, silicone waxes such as alkyl- or alkoxydimethicones having an alkyl or alkoxy chain ranging from 10 to 45 carbon atoms, poly(di)methylsiloxane esters which are solid at 30° C. and whose ester chain comprising at least 10 carbon atoms, di(1,1,1-trimethylolpropane) tetrastearate, which is sold or manufactured by Heterene under the name HEST 2T-4S; alkylated silicone acrylate copolymer waxes comprising at least 40 mole % of siloxy units having the formula (R₂R′SiO_{1/2})ₓ(R″SiO_{3/2})_{y}, where x and y have a value of 0.05 to 0.95, R is an alkyl group having from 1 to 8 carbon atoms, an aryl group, a carbinol group, or an amino group, R is a monovalent hydrocarbon having 9-40 carbon atoms, R″ is a monovalent hydrocarbon group having 1 to 8 carbon atoms, an aryl group such as those disclosed in U.S. patent application 2007/0149703, the entire contents of which is hereby incorporated by reference, with a particular example being C30-C45 alkyldimethylsilyl polypropylsilsesquioxane; and mixtures thereof.

According to preferred embodiments, the compositions of the present invention contain less than 1% wax.

According to preferred embodiments, the compositions of the present invention contain less than 0.5% wax.

According to preferred embodiments, the compositions of the present invention contain no wax.

If present, the wax or waxes may be present in an amount ranging from 1 to 30% by weight relative to the total weight of the composition, for example from 2 to 20%, and for example from 3 to 10%, including all ranges and subranges therebetween.

Aqueous Phase

The compositions of the present invention may also contain water. When the compositions of the present invention contain water, they are preferably in the form of an emulsion. Preferably, when the compositions of the present invention contain water, they are in the form of an oil-in-water emulsion (O/W) or a water-in-oil emulsion (W/O). Preferably, when in the form of an emulsion, the oil phase contains predominantly silicone oils (Si/W or W/Si emulsion) or hydrocarbon oils. When present, water is preferably present in an amount of from about 10% to about 80% by weight, preferably from about 20% to about 70% by weight, preferably from about 35% to about 65% by weight, including all ranges and subranges therebetween, all weights being based on the total weight of the composition.

Additional Additives

The composition of the invention can also comprise any additive usually used in the field under consideration. For example, dispersants such as poly(12-hydroxystearic acid), antioxidants, essential oils, sunscreens, preserving agents, fragrances, fillers, neutralizing agents, cosmetic and dermatological active agents such as, for example, emollients, moisturizers, vitamins, essential fatty acids, surfactants, silicone elastomers, thickening agents, gelling agents, particles, pasty compounds, viscosity increasing agents can be added. A non-exhaustive listing of such ingredients can be found in U.S. patent application publication no. 2004/0170586, the entire contents of which is hereby incorporated by reference. Further examples of suitable additional components can be found in the other references which have been incorporated by reference in this application. Still further examples of such additional ingredients may be found in the International Cosmetic Ingredient Dictionary and Handbook (9^{th} ed. 2002).

A person skilled in the art will take care to select the optional additional additives and/or the amount thereof such that the advantageous properties of the composition according to the invention are not, or are not substantially, adversely affected by the envisaged addition.

These substances may be selected variously by the person skilled in the art in order to prepare a composition which has the desired properties, for example, consistency or texture.

These additives may be present in the composition in a proportion from 0% to 99% (such as from 0.01% to 90%) relative to the total weight of the composition and further such as from 0.1% to 50% (if present), including all ranges and subranges therebetween.

Needless to say, the composition of the invention should be cosmetically or dermatologically acceptable, i.e., it should contain a non-toxic physiologically acceptable medium and should be able to be applied to the keratinous materials of human beings such as, for example, lips, skin or eyelashes.

In particular, suitable gelling agents for the oil phase include, but are not limited to, lipophilic or hydrophilic clays.

The term “hydrophilic clay” means a clay that is capable of swelling in water; this clay swells in water and forms after hydration a colloidal dispersion. These clays are products that are already well known per se, which are described, for example, in the book “Mineralogie des argiles”, S. Caillere, S. Henin, M. Rautureau, 2^{nd} edition 1982, Masson, the teaching of which is included herein by way of reference. Clays are silicates containing a cation that may be chosen from calcium, magnesium, aluminium, sodium, potassium and lithium cations, and mixtures thereof. Examples of such products that may be mentioned include clays of the smectite family such as montmorillonites, hectorites, bentonites, beidellites and saponites, and also of the family of vermiculites, stevensite and chlorites. These clays may be of natural or synthetic origin.

Hydrophilic clays that may be mentioned include smectite products such as saponites, hectorites, montmorillonites, bentonites and beidellite. Hydrophilic clays that may be mentioned include synthetic hectorites (also known as laponites), for instance the products sold by the company Laporte under the names Laponite XLG, Laponite RD and Laponite RDS (these products are sodium magnesium silicates and in particular sodium lithium magnesium silicates); bentonites, for instance the product sold under the name Bentone HC by the company Rheox; magnesium aluminium silicates, especially hydrated, for instance the products sold by the Vanderbilt Company under the names Veegum Ultra, Veegum HS and Veegum DGT, or calcium silicates, and especially the product in synthetic form sold by the company under the name Micro-cel C.

The term “lipophilic clay” means a clay that is capable of swelling in a lipophilic medium; this clay swells in the medium and thus forms a colloidal dispersion. Examples of lipophilic clays that may be mentioned include modified clays such as modified magnesium silicate (Bentone Gel VS38 from Rheox), and hectorites modified with a C₁₀ to C₂₂ fatty-acid ammonium chloride, for instance hectorite modified with distearyldimethylammonium chloride (CTFA name: disteardimonium hectorite) sold under the name Bentone 38 CE by the company Rheox or Bentone 38V® by the company Elementis.

In particular, among the gelling agents that may be used, mention may be made of silica particles. Preferably, the silica particles are fumed silica particles.

Suitable silicas include, but are not limited to, hydrophobic silicas, such as pyrogenic silica optionally with hydrophobic surface treatment whose particle size is less than 1 micron, preferably less than 500 nm, preferably less than 100 nm, preferably from 5 nm to 30 nm, including all ranges and subranges therebetween. It is in fact possible to modify the surface of silica chemically, by a chemical reaction producing a decrease in the number of silanol groups present on the surface of the silica. The silanol groups can notably be replaced with hydrophobic groups: a hydrophobic silica is then obtained. The hydrophobic groups can be:

trimethylsiloxyl groups, which are notably obtained by treatment of pyrogenic silica in the presence of hexamethyldisilazane. Silicas treated in this way are called “Silica silylate” according to the CTFA (6th edition, 1995). They are for example marketed under the references “AEROSIL R812®” by the company Degussa, “CAB-O-SIL TS-530®” by the company Cabot;

dimethylsilyloxyl or polydimethylsiloxane groups, which are notably obtained by treatment of pyrogenic silica in the presence of polydimethylsiloxane or dimethyldichlorosilane. Silicas treated in this way are called “Silica dimethyl silylate” according to the CTFA (6th edition, 1995). They are for example marketed under the references “AEROSIL R972®”, “AEROSIL R974®” by the company Degussa, “CAB-O-SIL TS-610®”, “CAB-O-SIL TS-720®” by the company Cabot.

Preferably, the gelling agent, if present, is present in the composition of the present invention in amounts of active material generally ranging from about 0.1% to about 10%, preferably from about 0.25% to about 5%, and more preferably from about 0.5% to about 3.5%, by weight, based on the total weight of the composition, including all ranges and subranges in between.

According to particularly preferred embodiments of the present invention, compositions of the present invention further comprise at least one surfactant present in an amount ranging from about 3% to about 10% by weight, at least one pigment in an amount ranging from about 10% to about 20% by weight, and at least one filler present in an amount ranging from about 0.5% to about 2% by weight, all weights being in relation to the total weight of the composition, including all ranges and subranges therebetween.

According to preferred embodiments of the present invention, methods of treating, caring for and/or making up a keratinous material by applying compositions of the present invention to the keratinous material in an amount sufficient to treat, care for and/or make up the keratinous material are provided. Preferably, “making up” the keratinous material includes applying at least one coloring agent to the keratinous material in an amount sufficient to provide color to the keratinous material.

According to yet other preferred embodiments, methods of enhancing the appearance of a keratinous material by applying compositions of the present invention to the keratinous material in an amount sufficient to enhance the appearance of the keratinous material are provided.

In accordance with the preceding preferred embodiments, the compositions of the present invention are applied topically to the desired area of the keratinous material in an amount sufficient to treat, care for and/or make up the keratinous material, to cover or hide defects associated with keratinous material, or to enhance the appearance of keratinous material. The compositions may be applied to the desired area as needed, preferably once daily, and then preferably allowed to dry before subjecting to contact such as with clothing or other objects. Preferably, the composition is allowed to dry for about 4 minutes or less, more preferably for about 2 minutes or less.

According to preferred embodiments of the present invention, methods of increasing strain sweep of a composition comprising combining at least one acid-functional silicone elastomer and at least one block copolymer in the composition in amounts and/or ratios indicated in the description above so as to increase strain sweep of the composition are provided. Preferably, strain sweep increase is manifested in improved stability of the composition, improved adhesion of the composition, or both as discussed above.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### Example 1—Inventive Composition

Sample Base Composition

| | |
|---|---|
| Surfactants | 3%-5% |
| Oil/Solvent | 5%-30% |
| Acid-Functional | 6% (active |
| Silicone elastomer | solids content) |
| Block Copolymer Such As Styrene-Containing | 8% (active |
| Polymer (e.g., Kraton materials) | solids content) |
| Filler | 0.1%-3% |
| Pigments | 10%-20% |
| Water Phase (film formers, preservatives, | 20%-50% |
| water-soluble solvents) | |
| Total | 100.0 |

### Example 2—Inventive and Comparative Compositions—Transfer Resistance

Invention compositions containing varying amounts of styrene-ethylene/butylene-styrene (SEBS) block copolymer and acid-functional silicone elastomer were prepared using the sample base composition of example 1.

The compositions were tested as follows in a multi-challenge wear test. A film was deposited onto a surface such as a black bykochart Black Scrub Panels P121-10N #5015 using a 1 mL drawdown bar. After drying over night at 37 C, the deposit was covered with artificial sebum or sweat. The film was wiped by hand with an absorbent material such as cotton. It was determined how much of the sample remained undisrupted on the scrub panel as well as how much of the sample was transferred to the absorbent material. 1 is total disruption and extreme transfer to absorbent material, 2 is most disruption with substantial transfer to absorbent material, 3 is half disruption and some transfer to the absorbent material, 4 is minimal disruption and minimal transfer to absorbent material and 5 is essentially no visual disruption (removal) and no visual transfer.

The table below sets forth the amount of SEBS block copolymer and functional silicone elastomer used in the different compositions, as well as the results of the testing.

| | | | Smudge | |
|---|---|---|---|---|
| | | Sweat | test: | Sebum |
| | Smudge | transfer | Sebum on | transfer |
| | test: sweat | on | abrasion | on |
| Formula | on abrasion | a cotton | paper | a cotton |
| Example 2A (2% acid- | 5 | 5 | 3 | 2 |
| functional silicone | | | | |
| elastomer + 2% SEBS) | | | | |
| Example 2B (5% acid- | 5 | 5 | 3 | 2 |
| functional silicone | | | | |
| elastomer + 2.5% SEBS) | | | | |
| Example 2C (5% acid- | 5 | 5 | 3 | 3 |
| functional silicone | | | | |
| elastomer + 5% SEBS) | | | | |
| Example 2D (5% acid- | 5 | 4 | 1 | 1 |
| functional silicone | | | | |
| elastomer) | | | | |
| Commercial Product | 3 | 3 | 3 | 3 |
| (containing silicone | | | | |
| resin) | | | | |

The testing showed that sebum resistance was improved using the combination of block copolymer and acid-functional silicone elastomer of the present invention. Acid-functional silicone elatomer, by itself, showed good sweat resistance properties. However, it showed poor sebum resistance properties. The commercially-available product showed lesser sweat resistance properties. In contrast, the invention combination of ingredients surprisingly showed both good sweat resistance properties and improved sebum resistance properties, with at least 5% of both the block copolymer and the acid-functional silicone elastomer being present demonstrating the best results for the properties tested which, in turn, demonstrate the best results related to improved adhesion to materials to which the compositions have been applied.

### Example 3—Inventive and Comparative Compositions—Strain Sweep

Strain sweep was determined by Rheometer (Discovery Hybrid Rheometer-3) for each formula. The experiment was conducted at 32° C. Angular frequency was fixed at 1 rad/s, while strain % was from 0.01% to 1000%.

| | Example 2A | Example 2B | Example 2C | Example 2D |
|---|---|---|---|---|
| Yield Stress | 0.58 | 7.7 | 7.14 | 0.17 |
| (Pa) - strain | | | | |
| sweep | | | | |
| Yield strain | 0.43 | 1.64 | 1.64 | 1.61 |
| (%) | | | | |
| Consistency | 44 | 508 | 343 | 11 |
| G* at 1% | | | | |
| strain (Pa) | | | | |
| G″ (Pa)at 300 | 6 | 14 | 9.96 | 1.06 |
| % strain | | | | |
| G′, G″ (Pa) at | 32, 30 | 496, 108 | 338, 58 | 10, 3 |
| 1% strain | | | | |
| Sol gel point | 1.85 | 5.21 | 5.6 | 20 |
| (%) | | | | |
| delta at 1% | 42 | 12 | 9.8 | 15 |
| strain | | | | |
| Dynamic | 30 | 108 | 58 | 3 |
| viscosity h ′at | | | | |
| 1% strain | | | | |
| Strain | N | Y | Y | Y |
| hardening | | | | |

Formula B showed the highest consistency and modulus. We observed strain hardening on Formula B and C and D. This suggested that strain hardening may correlate with higher concentration of acid-functional silicone elastomer, again correlating with improved adhesion to materials to which the compositions have been applied.

### Example 4—Inventive and Comparative Compositions—Coefficient of Friction

The coefficient of friction as a function of shear rate was measured by DHR-3 (Discovery Hybrid Rheometer). The experiment was conducted at 32° C. Axial force was fixed at 3 N±0.1 N. The mode was compression. The coefficient of friction was measured for 240 sec from 0.01 to 30 shear rate (/s) in a linear mode. Measuring coefficient of friction at a certain velocity by tribology is believed to be associated with sensory attributes.

It was observed that among formulae A, B, and C, there was no significant difference for friction. Formulae A, B and C generally showed higher friction relative to the commercially available product.

| Shear rate | Friction at 0.07/s | 1.1/s | 10/s |
|---|---|---|---|
| Example 2A | 0.11 | 0.24 | 0.31 |
| Example 2B | 0.12 | 0.24 | 0.33 |
| Example 2C | 0.12 | 0.24 | 0.33 |
| Commercial Product 1 | 0.13 | 0.28 | 0.29 |
| (containing MQ resin) | | | |
| Commercial Product 2 | 0.11 | 0.23 | 0.31 |
| (containing † propyl | | | |
| resin + † propyl resin | | | |
| wax) | | | |

What is claimed is:

1. A composition comprising at least one acid-functional silicone elastomer and at least one block copolymer which is a thermoplastic elastomer comprising styrene.

2. The composition of claim 1, wherein the composition is anhydrous.

3. The composition of claim 1, wherein the composition is in the form of an emulsion.

4. The composition of claim 1, wherein the composition further comprises at least one coloring agent.

5. The composition of claim 1, further comprising at least one volatile oil

6. The composition of claim 6, wherein the volatile oil is selected from the group consisting of cyclopentasiloxane, isododecane, and mixtures thereof.

7. The composition of claim 1, wherein the acid-functional silicone elastomer has a general formula: A-B-A or B-A-B, wherein each A independently comprises a polysiloxane moiety having at least two siloxy groups, wherein each B independently comprises a moiety, or a precursor thereof, having at least two carboxyl groups and wherein B is bonded to a silicon atom in A.

8. The composition of claim 1, wherein the block copolymer is styrene-ethylene/butylene-styrene.

9. The composition of claim 7, wherein the block copolymer is styrene-ethylene/butylene-styrene.

10. The composition of claim 1, wherein the block copolymer and acid-functional silicone elastomer are present in the compositions of the present invention in an active solid content weight ratio of about 3:1 to about 1:1.

11. The composition of claim 9, wherein the block copolymer and acid-functional silicone elastomer are present in the compositions of the present invention in an active solid content weight ratio of about 3:1 to about 1:1.

12. The composition of claim 1, wherein the block copolymer and acid-functional silicone elastomer each present in the compositions of the present invention in an active solid content of at least 2% by weight with respect to the total weight of the composition.

13. The composition of claim 11, wherein the block copolymer and acid-functional silicone elastomer each present in the compositions of the present invention in an active solid content of at least 2% by weight with respect to the total weight of the composition.

14. The composition of claim 1, wherein the block copolymer and acid-functional silicone elastomer each present in the compositions of the present invention in an active solid content of at least 5% by weight with respect to the total weight of the composition.

15. The composition of claim 11, wherein the block copolymer and acid-functional silicone elastomer each present in the compositions of the present invention in an active solid content of at least 5% by weight with respect to the total weight of the composition.

16. A method of making up a keratinous substrate comprising applying the composition of claim 1 to a keratinous substrate in an amount sufficient to makeup the keratinous substrate.
